(19) United States
(12) Patent Application Publication
(10) Pub. No.: US 2026/0295218 A1
(43) Pub. Date: Oct. 1, 2026

(54) SAFETY CLIP FOR A MEDICAL DEVICE AND METHOD OF USE

(71) Applicant: TELEFLEX MEDICAL LLC, Morrisville, NC (US)

(72) Inventors: Jacob Ammarell, Sinking Spring, PA (US); Jeffrey Kuehn, Schuylkill Haven, PA (US); Benjamin Horst, Lititz, PA (US)

(21) Appl. No.: 19/631,919

(22) Filed: Mar. 27, 2026

Related U.S. Application Data
(60) Provisional application No. 63/780,002, filed on Mar. 28, 2025.

(51) Int. Cl. A61M 25/06; A61M 25/09

(52) U.S. Cl. CPC A61M 25/0618; A61M 25/09

(57) ABSTRACT

The present invention describes a safety clip for a catheter insertion needle including a proximal portion including a first proximal wall connected to a second proximal wall at a proximal clip bend, the first proximal wall having two apertures. Further, when a first clip arm and a second clip arm are in an open configuration, the safety clip slides along the needle and the first distal wall and the second distal wall are spaced apart. Additionally, when the first clip arm and the second clip arm are in a closed configuration, the first proximal aperture and the second proximal aperture are configured to engage a swaged portion of the needle so the first distal wall and the second distal wall overlap to cover a tip of the needle. The first clip arm and the second clip arm do not intersect in either the open configuration or the closed configuration.

### CROSS REFERENCE TO RELATED APPLICATIONS

This application claims the benefit of priority to U.S. Provisional Application 63/780,002, filed Mar. 28, 2025, the entire contents of which are incorporated by reference herein.

### FIELD OF THE INVENTION

The present disclosure relates to safety devices and methods of their use, and in particular, to a safety clip configured to slide on a needle, clip securely on the needle, and cover the tip of the needle to provide sharps protection.

### BACKGROUND

In clinical practice, injuries from needles are dangerous because of blood and infective agents that contaminate the used needle. These injuries, which are occupational hazards of the medical profession, have resulted in inadvertent transmission of infective agents such as hepatitis C and human immunodeficiency virus. Sharps collection boxes, gloves and various safety practices are used to mitigate injuries arising from used needles, and to prevent infections from the needles. However, the presently available safety devices for protecting against injuries and infections resulting from needles are not completely fail-safe.

Moreover, radial artery catheters often have integrated needles which many existing needle safeties do not work with. Furthermore, conventional safety clips that work with guidewires are often too large and thus cause the needle housing to be too big or bulky, which provides for poor device ergonomics and increases the likelihood of adverse behavior of the safety clip during catheter insertion. Therefore, the present disclosure addresses these unmet needs by providing a new safety clip for a needle that is compatible for use with a guidewire, provides for improved ergonomics, minimizes the risk of poor safety clip performance, and is easy to manufacture.

### SUMMARY

As used herein, including the appended claims, the singular forms of words such as “a,” “an,” and “the” include their corresponding plural references unless the context clearly dictates otherwise. All references cited herein are incorporated by reference to the same extent as if each individual publication, patent, and published patent application, as well as figures and drawings in said publications and patent documents, was specifically and individually indicated to be incorporated by reference.

The present disclosure solves the foregoing problems and relates to a safety clip for a needle and method of use of the safety clip to prevent injury. For example, a safety clip may include a proximal portion having a first proximal wall directly connected to a second proximal wall at a clip bend, the first proximal wall defining a first proximal aperture and the second proximal wall defining a second proximal aperture, the second proximal wall being more proximal than the first proximal wall. The safety clip may also have a first distal portion comprising a first distal wall, the first distal wall defining a first distal aperture for receiving the needle. Further, the safety clip may include a first clip arm extending from the first distal portion to the first proximal wall and including a medial aperture for receiving the needle. Additionally, the safety clip may have a second distal portion including a second distal wall, which defines a second distal aperture for receiving the needle.

Moreover, the safety clip may have a second clip arm extending from the second distal portion to the second proximal wall. The first clip arm and the second clip arm may be in contact with the needle, and the first clip arm and the second clip arm may be configured to flex between a first configuration and a second configuration to move the safety clip relative to the needle. When the first clip arm and the second clip arm are in the first configuration, the safety clip may be able to slide along a length of the needle and the first distal wall and the second distal wall are spaced apart and when the first clip arm and the second clip arm are in the second configuration, the second proximal aperture may be configured to engage a swaged portion of the such that the first distal wall and the second distal wall overlap to cover a tip of the needle. The first clip arm and the second clip arm may also not intersect in either the first configuration or the second configuration.

The described implementations of the invention may also include one or more of the following features. For example, the first and/or second clip arms may be at a rest state in the closed configuration. Further, a first distal slot may extend through a portion of the first distal wall and the first distal face, the first distal slot configured to receive a guidewire of the needle. There may also be a second distal slot extending through a portion of the second distal wall and the second distal face, which may be configured to receive the guidewire of the needle. Also, the first distal slot and the second distal slot may be configured to allow a guidewire to retract back inside the tip of the needle when the first clip arm and the second clip arm are in the closed configuration.

The first distal slot and the second distal slot may be configured to allow the first clip arm and the second clip arm to move to the closed configuration even with the guidewire advanced beyond the tip of the needle. Additionally, the safety clip may include a one-piece body. Moreover, the first distal portion of the safety clip may include a first curved elbow connecting the first clip arm to the first distal wall, and the second distal portion of the safety clip may include a second curved elbow connecting the second clip arm to the second distal wall. Further, the first and second curved elbows may be configured to contact a bump portion within a hub of a medical device to prevent removal of the safety clip from inside the hub when in the open configuration.

The safety clip may also be configured to be removed from the hub of the medical device when the first clip arm and the second clip arm are in the closed configuration. In that case, the medical device may be a catheter, which includes a catheter hub. Implementations of the invention may also include a bump on the hub of the medical device configured to prevent the safety clip from being removed from the hub in the open configuration and configured to allow the safety clip to be removed from the hub in the closed configuration. Moreover, the safety clip may exert a first force on the needle when the first clip arm and the second clip arm are in the open configuration, which tends to return the safety clip back to the closed configuration.

Additionally, the invention may also include a keeper disposed between the first and second proximal walls, where the keeper may be configured to provide frictional resistance against an inner surface of a hub of a medical device to retain the safety clip inside the catheter hub. The keeper may be made of an elastomeric material, preferably silicone and may include a through-hole configured to slidably receive the needle. Further, a diameter of the through-hole may be greater than a diameter of the first proximal aperture and a diameter of the second proximal aperture. The keeper may also be configured to prevent the safety clip from rotating around a longitudinal axis of the catheter as the safety clip slides down the length of the needle.

The first proximal aperture may also be coaxial with the second proximal aperture and the first distal face and the second distal face may be configured to press against the needle when the first and second clip arms are in the open configuration. Furthermore, the second distal bend may be configured to abut the first distal wall when the first and second clip arms in the closed configuration. Finally, a width of the first distal slot may be greater than a width of the first medial slot, and a width of the second distal slot may be greater than a width of the second medial slot.

The invention may also relate to a method of preventing injury while using a medical device. This method may involve using a safety clip which may have a proximal portion comprising a first proximal wall directly connected to a second proximal wall at a proximal clip bend, the first proximal wall defining a first proximal aperture and the second proximal wall defining a second proximal aperture, and the second proximal wall being more proximal than the first proximal wall. Additionally, the safety clip may have a first distal portion comprising a first distal wall, and the first distal wall defining a first distal slot. Further, the safety clip may have a first clip arm extending from the first distal portion to the first proximal wall and include a first medial elongated slot and a second distal portion comprising a second distal wall, and the second distal wall may define a second distal slot. The safety clip used may also include a second clip arm extending from the second distal portion to the second proximal wall and the first clip arm and the second clip arm may be configured to flex between a open configuration and a closed configuration; and wherein the first clip arm and the second clip arm do not cross in either the open configuration or the closed configuration.

Additionally, the method may include using a needle with a swaged portion; and a hub of the medical device comprising a bump separating the hub into a proximal cavity and a distal cavity. Further, the method may include the step of positioning the proximal cavity of the hub around the needle and the safety clip and moving the hub proximally until the first distal wall and the second distal wall of the safety clip extend past the bump and within the distal cavity of the hub. The method may also include sliding the hub and the safety clip in a proximal direction along a length of the needle such that the first clip arm and the second clip arm transition from the closed configuration to the open configuration such that the first distal wall and the second distal wall are spaced apart and the first distal wall and the second distal wall engage with the hub in the distal cavity. Finally, the method may include further sliding the hub and the safety clip in the proximal direction along the length of the needle until the medical device is installed.

The described method may also include one or more of the following features. For example, the method may further include moving the hub and the safety clip in a distal direction along the length of the needle until the second proximal aperture is at a proximal end of the swaged portion of the needle and the first clip arm and the second clip arm transition from the open configuration to the closed configuration. Additionally, the method may include sliding the hub in the proximal direction so that the bump is able to pass the first distal wall and the second distal wall of the safety clip and off of the tip of the needle. Further, the hub may be a catheter hub and the medical device may be a catheter. Additionally, the safety clip may further comprise a keeper configured to maintain the clip inside the catheter hub until the first proximal aperture is engaged with the swaged portion of the needle in the closed configuration. Finally, the safety clip may further comprise a keeper configured to prevent the safety clip from rotating around a longitudinal axis of the catheter as the safety clip slides down the length of the needle.

### BRIEF DESCRIPTION OF THE DRAWINGS

FIG. 1 discloses a side view of the safety clip within a catheter hub of a catheter insertion device according to aspects of the disclosure.

FIG. 2 shows a perspective view of the safety clip as seen in FIG. 1.

FIG. 3 shows a side plan view of the safety clip seen in FIG. 1 in the first, open configuration.

FIG. 4 shows a side plan view of the safety clip seen in FIG. 1 in the second, closed configuration.

FIG. 5 shows a perspective view of the safety clip seen in FIG. 1 in the first, open configuration.

FIG. 6 shows a perspective view of the safety clip seen in FIG. 1 in the second, closed configuration.

FIG. 7 shows a perspective view of the safety clip seen in FIG. 1 in the first configuration.

FIG. 8 shows a perspective view of the safety clip seen in FIG. 1 in the second configuration.

FIG. 9 shows a top plan view of the safety clip seen in FIG. 1 in the first configuration.

FIG. 10 shows a side view of the safety clip seen in FIG. 1 in the first configuration prior to activation.

FIG. 11 shows a side view of the safety clip seen in FIG. 1 in between the first configuration and the second configuration during activation.

FIG. 12 shows a side view of the safety clip seen in FIG. 1 in the second configuration after activation.

FIG. 13 shows a top plan view of the safety clip seen in FIG. 1 in the first configuration.

FIG. 14 shows a perspective view of the safety clip seen in FIG. 1 in an intermediate configuration.

FIG. 15 shows a perspective view of an embodiment of a safety clip according to aspects of the disclosure in the first configuration.

FIG. 16 shows a perspective view of the safety clip seen in FIG. 14 in the second configuration.

FIG. 17 shows a side plan view of the safety clip seen in FIG. 14 in the first, open configuration.

FIG. 18 shows a side plan view of the safety clip seen in FIG. 14 in the second, closed configuration.

FIG. 19 shows a side view of the safety clip seen in FIG. 14 with a keeper in the first configuration in a catheter hub prior to activation.

FIG. 20 shows a side view of the safety clip seen in FIG. 14 with a keeper in between the first configuration and the second configuration in a catheter hub during activation.

FIG. 21 shows a side view of the safety clip seen in FIG. 14 with a keeper in the second configuration in a catheter hub after activation.

FIG. 22 shows a perspective view of an embodiment of a safety clip according to aspects of the disclosure in the first configuration.

FIG. 23 shows a perspective view of the safety clip seen in FIG. 21 in the second configuration.

FIG. 24 shows a side plan view of the safety clip seen in FIG. 21 in the first, open configuration.

FIG. 25 shows a side plan view of the safety clip seen in FIG. 21 in the second, closed configuration.

FIG. 26 shows a side view of the safety clip seen in FIG. 21 with a keeper in the first configuration in a catheter hub prior to activation.

FIG. 27 shows a side view of the safety clip seen in FIG. 21 with a keeper in between the first configuration and the second configuration in a catheter hub during activation.

FIG. 28 shows a side view of the safety clip seen in FIG. 21 with a keeper in the second configuration in a catheter hub after activation.

FIGS. 29A-29C show perspective views of a safety clip according to various embodiments.

### DETAILED DESCRIPTION

As will be discussed in detail herein, the present disclosure describes a safety clip to provide sharps protection in a medical device such as an over-the-needle catheter insertion device, such as a catheter insertion device operable to provide continuous pulsatile blood flashback, a peripheral IV catheter, a catheter over needle assembly that may be used for vessel access prior to insertion of a PICC (Peripherally Inserted Central Venous Catheter) or CVC (Central Venous Catheter) , or other vascular access devices. In particular, the safety clip is configured to provide sharps protection by sliding along a needle with a portion of the medical device, such as an over-the-needle catheter. The safety clip is configured to activate when it reaches the beveled tip of a needle and close around the tip such that when the needle is ready to be removed from the insertion site of the patient, the needle is covered.

In referencing the Figures and throughout the disclosure, like reference numerals (e.g., 300, 300’, 300’’) are used to identify like components. For brevity, the description of various components may not be repeated and will be understood to apply to all components unless the differences are otherwise described.

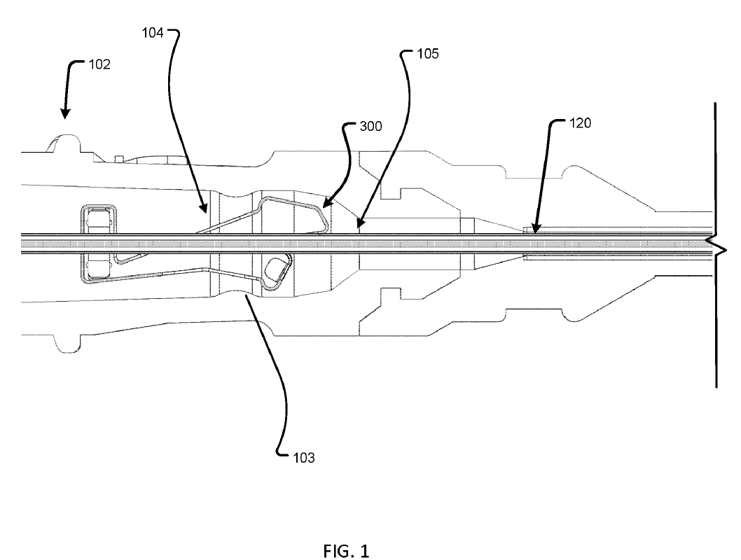

As seen in FIG. 1, a safety clip 300 may be used in a medical device which utilizes a needle 120 such as a catheter insertion device, a guidewire advancement device, syringe device, or any other device known to those skilled in the art.

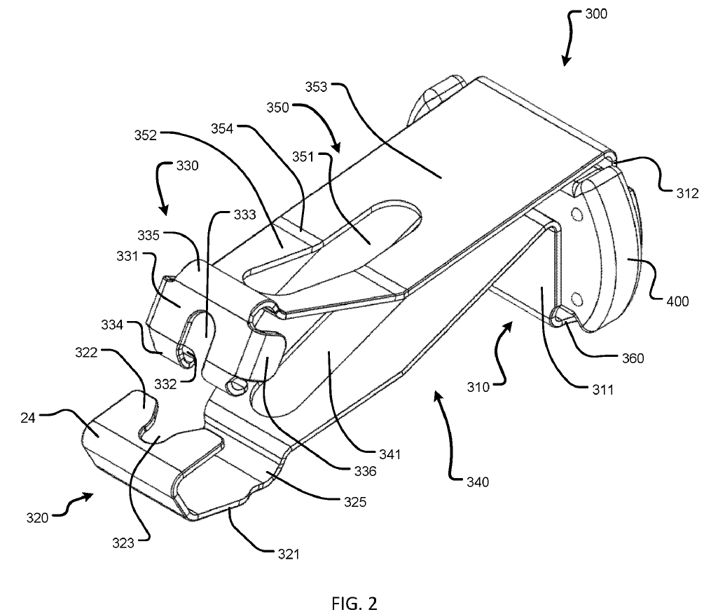

Referring to FIG. 2, a safety clip 300 is shown which may include a proximal portion 310 with a first proximal wall 311 directly connected to a second proximal wall 312 at a proximal clip bend 360. This proximal clip bend 360 may have a single curve, may be rectilinear, or may otherwise be any shape known to those skilled in the art capable of providing an elastic force. The first proximal wall 311 may have a first proximal aperture 313 and the second proximal wall 312 may have a second proximal aperture 314. In some implementations, the first proximal aperture 313 has an oblong or oval shape, and the second proximal aperture 314 has a round or circular shape. Further, the second proximal wall 312 may be located more proximal than the first proximal wall 311. In this manner, a needle of a medical device may slide through the first proximal aperture 313 and the second proximal aperture 314.

Additionally, the safety clip 300 may have a first nose or distal portion 320 with a first distal wall 321. The first distal wall 321 may be connected to a first distal face 322 by a first distal curved surface 324. The first distal face 322 may include a first distal slot 323 for receiving a portion of a needle and/or a guidewire. The safety clip 300 may also have a second nose or distal portion 330 having a second distal wall 331 connected to a second distal face 332 by a second distal curved surface 334. Extending laterally from the second distal wall 331 may be a pair of lateral walls or wings 336 configured to enhance sharps protection from the side of the safety clip 300. The first distal wall 321 may also include a pair of lateral walls and/or both the first distal wall 321 and the second distal wall 331 may include a pair of lateral walls. Further, a first distal slot 323 may extend through a portion of the first distal face 322, the first distal slot 323 is configured to receive a portion of the guidewire and/or the needle.

The safety clip 300 may have a second distal portion 330 with a second distal wall 331, the second distal wall 331 having a second distal slot 333 for receiving a portion of the guidewire and/or the needle, such as the needle tip. For instance, the second distal slot 333 may extend through a portion of the second distal wall 331 and the second distal face 332, which may be configured to receive the guidewire of the needle. The first distal wall 321 and the second distal wall 331 may have various shapes. For example, the first and second distal walls may be angular, curved, or flat.

The safety clip 300 may also include a first flexible clip arm 340 which may extend from the first distal portion 320 to the first proximal wall 311 and may include a first elongated medial slot 341 for receiving the needle 120. The safety clip 300 also can include a second flexible clip arm 350 extending from the second distal portion 330 to the second proximal wall 312, where the first flexible clip arm 340 and the second flexible clip arm 350 are configured to contact a needle. The second clip arm 350 may also include a second elongated medial slot 351 configured to receive a second portion of the needle. In this arrangement, the first distal portion 320 is more distal than the second distal portion 330 so that first distal wall 321 overlaps with the second distal wall 331 when the safety clip 300 is in its closed position.

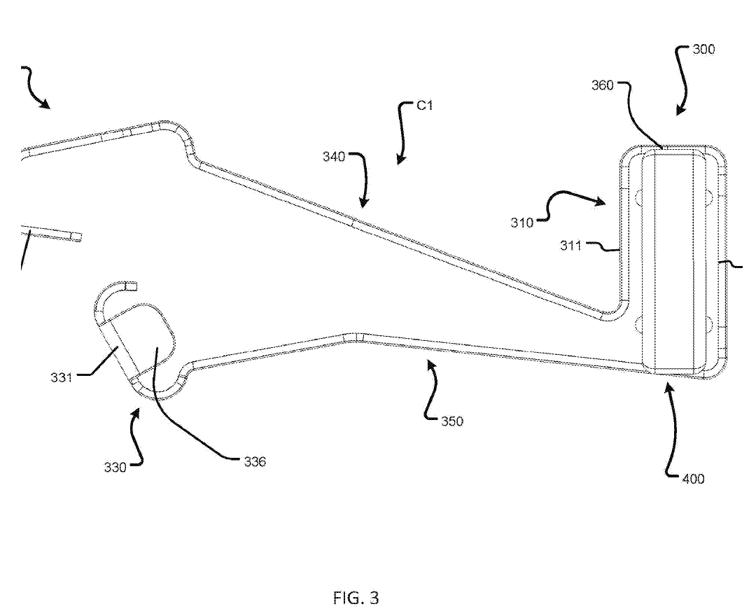

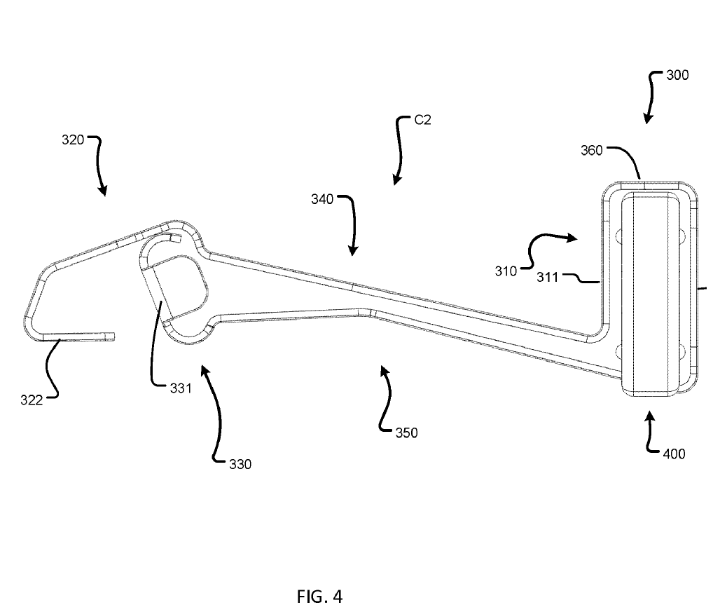

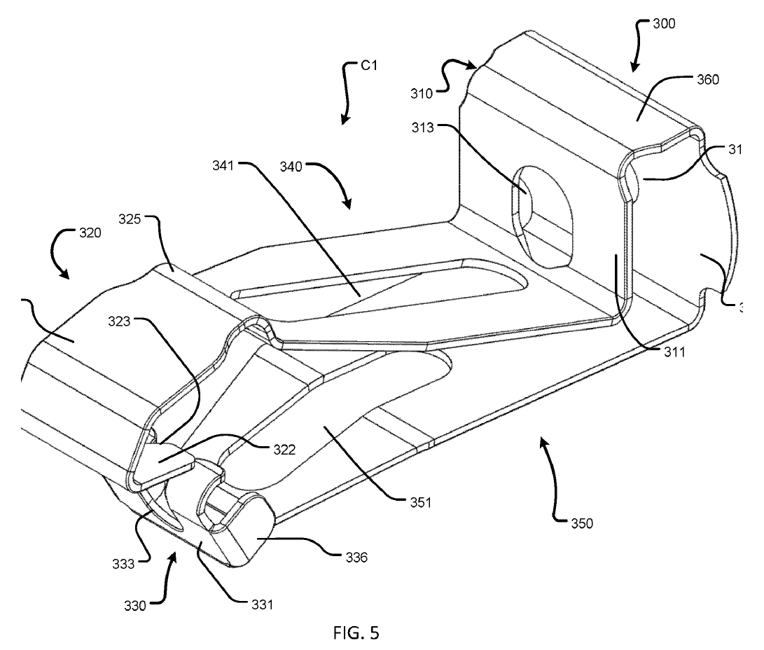

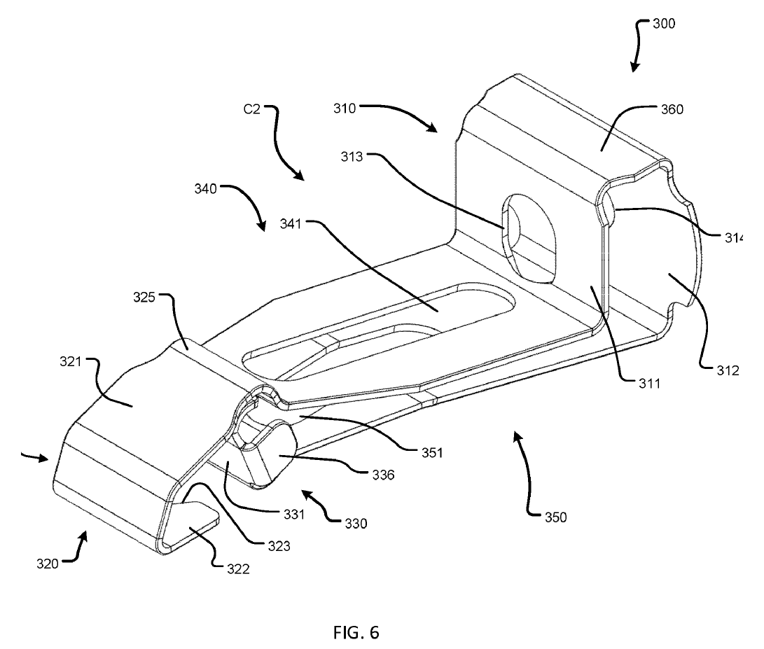

As seen in FIGS. 3-6, this design allows for the safety clip 300 to elastically move between two positions: a first, open configuration C1, and a second, closed configuration C2. It is important to note that, at rest, the safety clip 300 is configured so the first clip arm 340 and the second clip arm 350 are biased toward the closed configuration C2. Thus, the first clip arm 340 and/or the second clip arm 350 may be configured to press against a needle when in the closed configuration C2. In this way, the first clip arm 340, the second clip arm 350, or both may be configured to press against a needle and/or a guidewire when in the closed configuration C2 and provide sharps protection from the needle. Additionally, the first proximal aperture 313 may be coaxial with the second proximal aperture 314 and the first distal face 322 and the second distal face 332 may be configured to press against the needle and/or the guidewire when the first clip arm 340 and the second clip arm 350 are in the open configuration. Furthermore, the second distal curved surface 334 may be configured to abut the first distal wall 321 when the first clip arm 340 and the second clip arm 350 in the closed configuration. Moreover, the first distal slot 323 may have a first portion that is greater than a width of the first medial slot and a second portion that is less than a width of the first medial slot, and a width of the second distal slot 333 may be less than a width of the second medial slot. For example, the second distal slot 333 may be configured, such as in size and shape, to permit the guidewire to pass therethrough while simultaneously preventing the needle from passing therethrough. Moreover, when the safety clip 300 is in the closed configuration C2, the first distal curved surface 324 may fully cover the tip of the needle to prevent the needle from protruding from the safety 300 even if the safety clip 300 flexes from being pushed on after activation.

As a result, the first flexible clip arm 340 and the second flexible clip arm 350 may be configured to flex between the first, open configuration C1 and the second, closed configuration C2 to move the safety clip 300 relative to the needle. Further, the first flexible clip arm 340 and the second flexible clip arm 350 to move from the open configuration C1 to the closed configuration C2 as the first flexible clip arm 340 and the second flexible clip arm 350 are biased toward the closed configuration C2 in the rest state of the safety clip 300. Additionally, the first flexible clip arm 340 and the second flexible clip arm 350 are able to move to the closed configuration C2 with or without the guidewire being advanced beyond the tip of the needle. In some implementations, the safety clip 300 may be configured to move to an intermediate configuration C3 in which the first flexible arm 340 is moved to a partially closed configuration, due to the guidewire being advanced, and the second flexible arm 350 is moved to the closed configuration as described herein.

By way of example, referring back to FIG. 1, the catheter 101 may include a bump or protrusion 103 configured so that the internal diameter of the catheter hub 102 (i.e., at the protrusion 103) interferes with the safety clip 300 when the safety clip 300 is in its open configuration C1 and does not interfere with the safety clip 300 when the safety clip 300 is in its closed configuration C2. This feature also allows the catheter 101 to move simultaneously with the safety clip 300 when the user inserts the catheter 101 into a vessel of the patient. As such, the protrusion 103 may define the hub 102 into a proximal cavity 104 and a distal cavity 105.

That is, the safety clip 300 is in the open configuration C1 where its first distal wall 321 and the second distal wall 331 are spread apart at a radial distance beyond the internal diameter of the catheter hub 102 at the protrusion 103. When the safety clip 300 reaches the end of the needle 120, the safety clip 300 flexes to the closed configuration C2 compressing the first distal wall 321 and the second distal wall 331 together in such a way to allow the catheter 101 to freely move with respect to the needle 120, the safety clip 300, and the medical device. Thus, as the catheter 101 is inserted into a blood vessel of a patient the safety clip 300 will also cover the needle tip to protect the user from injury.

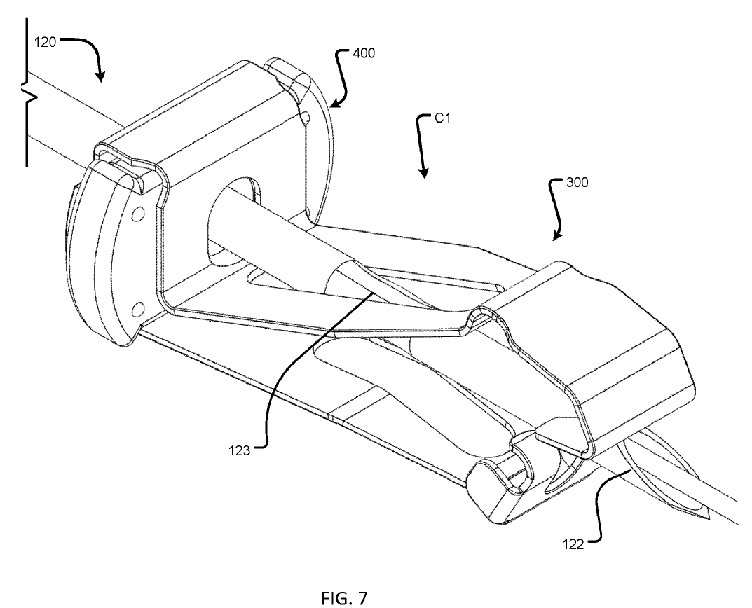

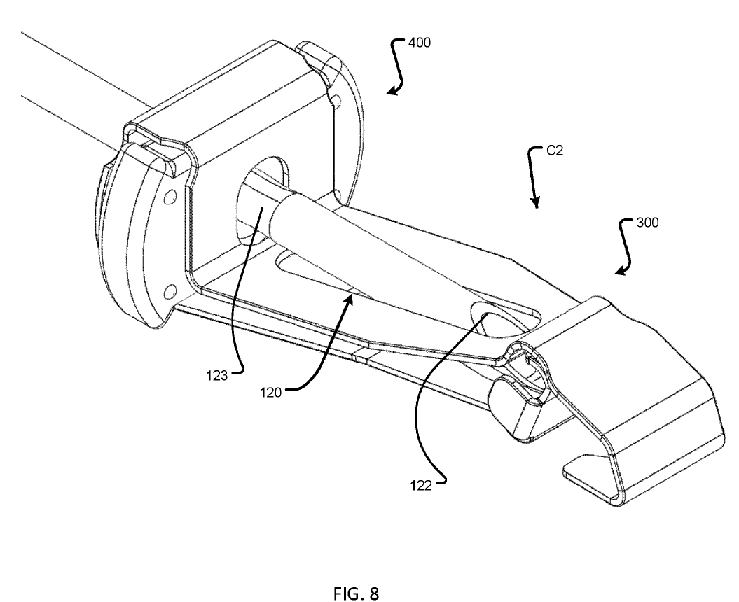

This flexing movement of the first and second flexible arms toward the closed configuration occurs when the first distal wall 321 and the second distal wall 331 of the safety clip 300 reach the needle tip 122 and the first proximal aperture 313 reaches a swaged portion 123 of the needle 120. Best seen in FIGS. 7 and 8, this swaged portion 123 is configured to be in an oval shape and prevents the safety clip 300 from being pulled easily off the needle tip 122 as the second proximal aperture 314 of the safety clip 300 has a smaller diameter than the major outer diameter of the swaged portion.

For example, when the first flexible clip arm 340 and the second flexible clip arm 350 are in the open configuration C1, the safety clip 300 may be able to slide along a length of the needle 120 and the first distal wall 321 and the second distal wall 331 are spaced apart. Additionally, when the first flexible clip arm 340 and the second flexible clip arm 350 are in the closed configuration C2, the second proximal aperture 314 may be configured to engage a swaged portion 123 of the needle. Further, in the closed configuration C2, the first distal wall 321 and the second distal wall 331 overlap to cover the tip 122 of the needle 120 as the first flexible clip arm 340 and the second flexible clip arm 350 move to the rest state of the safety clip 300 in the second configuration C2. Moreover, the first distal slot 323 and the second distal slot 333 may be configured to allow the first flexible clip arm 340 and the second flexible clip arm 350 to move to the closed configuration C2 with a guidewire advanced beyond the tip of the needle and may allow a guidewire to be retracted while in the closed configuration C2.

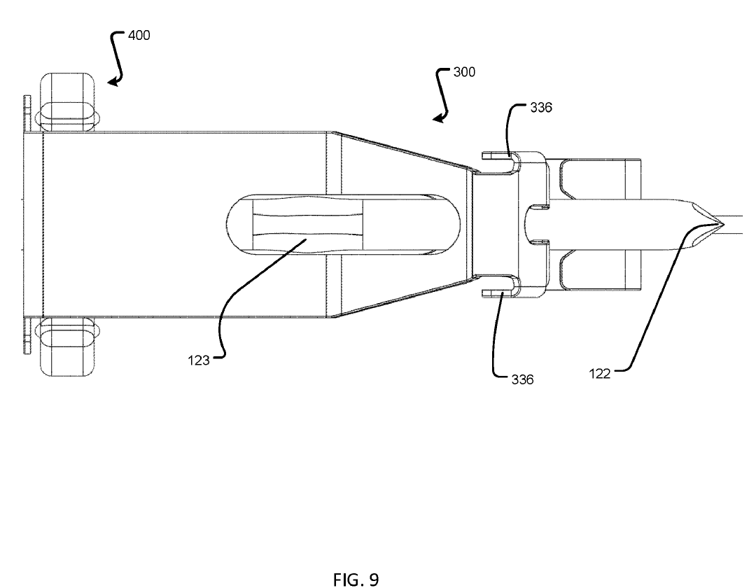

Thus, due to the first flexible clip arm 340 and the second flexible clip arm 350 being biased toward the closed configuration C2, the first distal wall 321 and the second distal wall 331 may be configured to cover a needle tip 122 to protect the user from injury. As seen in FIGS. 1-13, to form such a cover, the first distal wall 321 is more distal than the second distal wall 331. However, in other aspects of the invention, the second distal wall 331 may be more distal than the first distal wall 321. The first flexible clip arm 340 and the second flexible clip arm 350 do not cross each other in either the open configuration C1 or the closed configuration C2.

Accordingly, this design allows the safety clip 300 to easily be made in one piece. The safety clip 300 may be made by any known manufacturing method known to those skilled in the art such as 3D printing, casting, forging, die forming, and/or the like. Additionally, the clip may be made of any flexible, resilient or elastic material, such as a metal including aluminum or stainless steel, or plastic, capable of providing enough elastic force for the safety clip 300 to move between the open configuration C1 and the closed configuration C2. For example, the safety clip 300 may be manufactured of a single piece of metal, stamped, and then bent into shape.

Additionally, the safety clip 300 may include a one-piece body. Moreover, the first distal portion 320 of the safety clip 300 may include a first curved elbow 325 connecting the first clip arm 340 to the first distal wall 321, and the second distal portion 330 of the safety clip 300 may include a second curved elbow 335 connecting the second clip arm 350 to the second distal wall 331. The second clip arm 350 may include a first medial plane surface 352 and a second medial plane surface 353 connected by a medial bend 354. This arrangement allows for the first clip arm 340 and the second clip arm 350 to substantially overlap and provide sharps protection when in the second configuration C2. Further, the first and second curved elbows (325, 335) may be configured to contact a bump 103 portion within a hub of a medical device to prevent removal of the safety clip 300 from inside the hub when in the open configuration. The safety clip 300 may also be configured to be removed from the hub of the medical device when the first clip arm 340 and the second clip arm 350 are in the closed configuration.

In another aspect, such as seen in FIGS. 1 and 10-14, the safety clip 300 may include a flexible keeper 400, made of silicone or another elastomeric material between the first proximal wall 311 and the second proximal wall 312. The operation of the safety clip 300 and the structure of the safety clip 300 with and without the keeper 400 are substantially the same. However, the keeper 400 may be configured to ensure the safety clip 300 remains properly retained and oriented within the catheter hub 102 as the catheter 101 is inserted into the vessel of the patient and removed from the catheter insertion device 100. The keeper 400 may thus be configured to provide frictional resistance against an inner surface of the catheter hub 102 to prevent the safety clip 300 from pulling out of the hub 102 absent application of a threshold force.

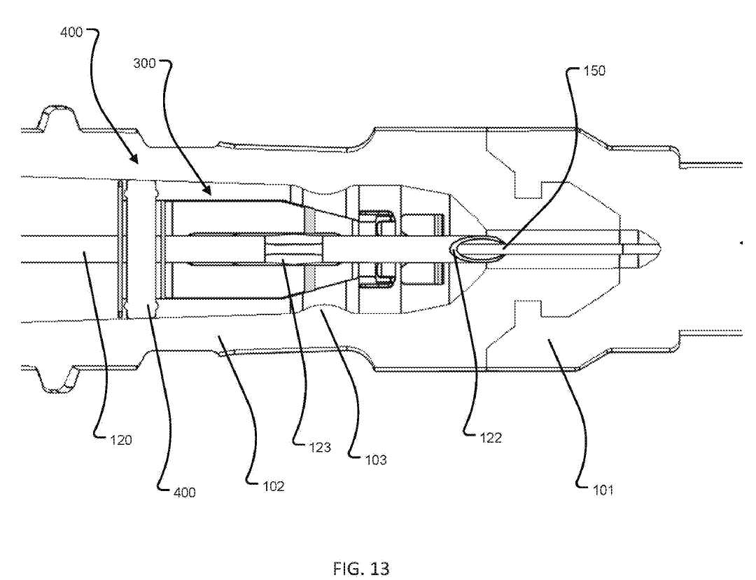

For this purpose, the catheter hub 102 initially has a larger inner diameter than the outer diameter of the keeper 400, but the inner diameter of the catheter hub 102 slowly decreases in diameter as the keeper 400 is pushed into the catheter hub 102 until the outer diameter of the keeper 400 is slightly larger than the inner diameter of the catheter hub 102, as seen for example in FIG. 13. This arrangement creates friction or drag between the keeper 400 and the catheter hub 102 to help ensure the safety clip 300 stays with the catheter 101 until the safety clip 300 can activate towards its closed configuration C2 to cover the needle tip 122 as the catheter 101 is being inserted into a vessel of a patient and the medical device is being removed from the catheter 101.

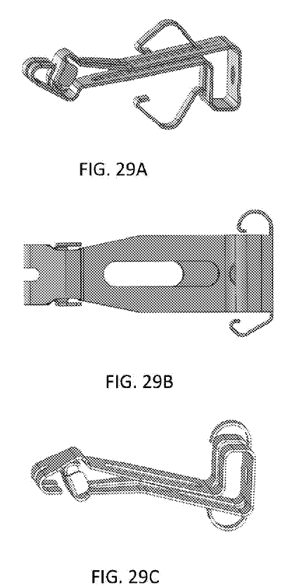

The keeper 400 may be of one piece that fits within the space between the first proximal wall 311 and the second proximal wall 312 of the safety clip 300 as seen in FIGS. 7-13 or could include projections integral with the safety clip 300 extending from the first proximal wall 311 and/or second proximal wall 312 of the safety clip 300 as seen in FIGS. 29A-29C. It should be noted that the force to pull the swaged portion 125 of the needle 120 through the internal diameter of the second proximal aperture 314 is much greater than the force to pull the keeper 400 from the internal diameter of the catheter 101.

The keeper 400 may also include a through-hole configured to slidably receive the needle. Further, a diameter of the through-hole may be greater than a diameter of the first proximal aperture 313 and a diameter of the second proximal aperture 314. Additionally, in embodiments where the safety clip 300 uses a keeper 400, the friction or drag force of the keeper 400 should not be large enough so that it would pull on the catheter 101 during removal of the medical device after the catheter 101 has been fully inserted into the blood vessel of the patient. However, a person skilled in the art would be able to determine through routine experimentation the desired interference fit and material capable of creating the desired results of the keeper 400 as described herein.

To assemble the catheter insertion device 100 with the safety clip 300 the keeper 400 is pushed into the space formed by the proximal walls 311, 312 and bend 360 then a tool is used to spread open the first clip arm 340 and the second clip arm 350. The back end of the needle 120 is then slid into the safety clip 300. In this way, the back end of the needle first slides past the first distal slot 323, then past the second distal slot 333, then through the first elongated medial slot 341, past the second elongated medial slot 351, through the first proximal aperture 313, and finally through the second proximal aperture 314 of the safety clip 300. The safety clip 300 will slide along the length of the needle 120 and will rest in the center of the length of the needle 120.

Here, the needle 120 and safety clip 300 and keeper 400 assembly is removed from the tool used to spread open the first clip arm 340 and the second clip arm 350. At this point, the needle 120 is glued, or otherwise affixed to the needle hub. A catheter 101 may then be slid over the needle tip 122 of the catheter insertion device 100 which causes the first curved elbow 325 and the second curved elbow 335 to contact the protrusion 103 of the catheter 101. Pushing on the catheter 101, slides the safety clip 300 along the needle 120 until the safety clip 300 contacts the needle hub 121. At this point, the needle hub 121 pushes back onto the safety clip 300 and causes the arms of the safety clip 300 to elastically compress until the first distal wall 321 and the second distal wall 331 have moved past the protrusion 103 at which point the safety clip 300 uncompresses back to its original geometry. Thereafter, the safety clip 300 will move simultaneously with the catheter 101 along the length of the needle 120 until the safety clip 300 activates. This structure of the safety clip 300 thus creates an over the needle catheter 101 that conceals the safety clip 300 during catheter insertion.

Then, after preparing the patient for needle insertion, the user (e.g., practitioner or clinician) may insert the needle 120 attached to a distal end of a medical device into the vessel of the patient. From here, the user may advance the guidewire, from a location where a guidewire tip 151 is within the needle 120, to a second position, where the guidewire tip 151 is advanced into a vessel of the patient.

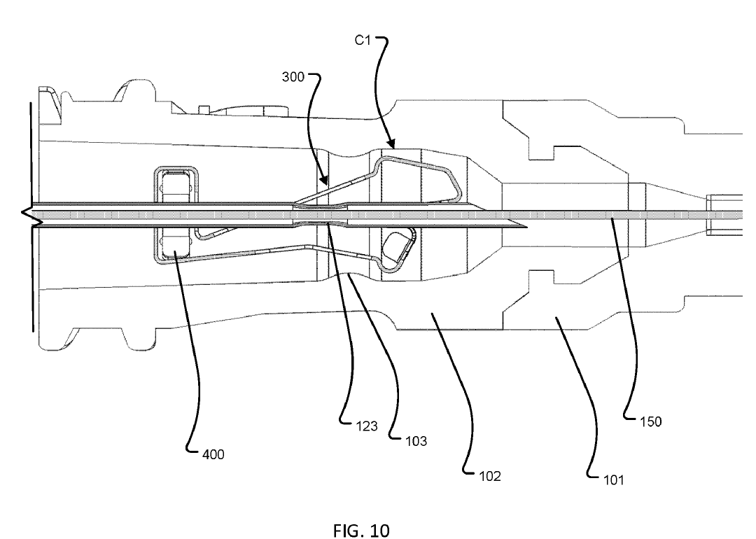

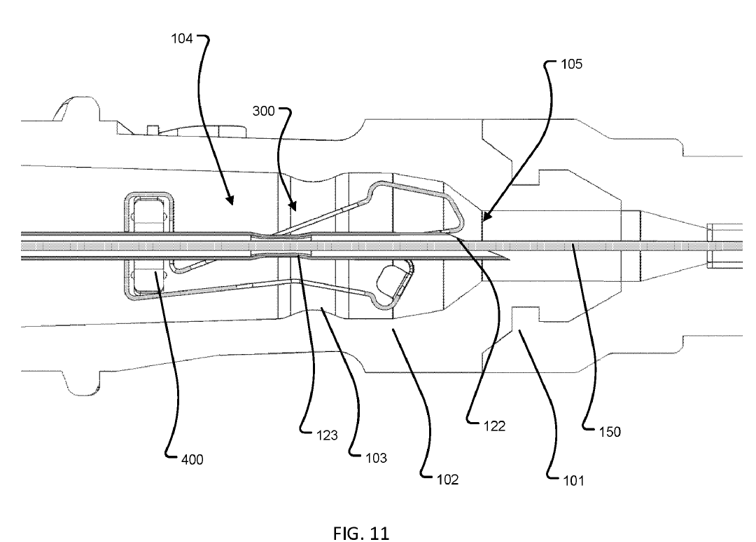

From here, the user may advance the catheter 101 forward to insert it into the vessel by pushing the catheter hub 102 forward over the needle until the catheter hub 102 is fully advanced into the insertion site. As the user moves the catheter 101 forward, a safety clip 300 within the catheter 101 will slide along the length of the needle 120. At this point, the needle 120 is retracted to remove it from the catheter 101. As the needle 120 is retracted and pulled fully out of the catheter hub, the needle tip 122 will be retracted until the arms of the safety clip 300 are able to activate and the distal walls (321, 331) will begin to close around the needle tip 122 to protect the user from injury. For example, when the first clip arm 340 and the second clip arm 350 are in the first configuration C1, the safety clip 300 may be able to slide along a length of the needle 120 and the first distal wall 321 and the first distal wall 331 are spaced apart. Further when the first clip arm 340 and the second clip arm 350 are in the first configuration C1, the first curved elbow 325 and the second curved elbow 335 are engaged with the protrusion 103 of the catheter hub 102 as the catheter 101 is inserted into the vessel of the patient. When the user begins to retract the needle after catheterization, for example, after the catheter is fully advanced into the insertion site, the needle will be moved proximally relative to the safety clip 300 to a point as seen in FIG. 10. At this point, the safety clip 300 is still in the open configuration C1. However, as the user continues to retract the needle, the safety clip 300 will begin to activate at which point, the first distal wall 321 and the second distal wall 331 are located approximately at the beginning of the beveled tip 122 of the needle, as seen in FIG. 11.

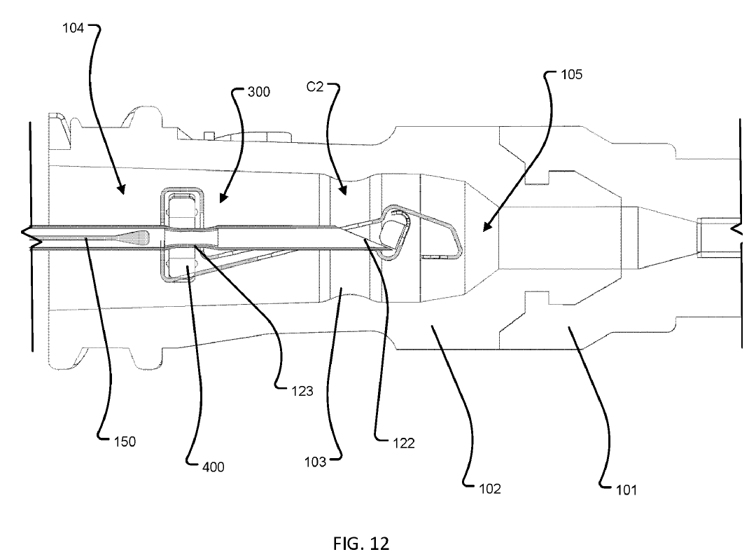

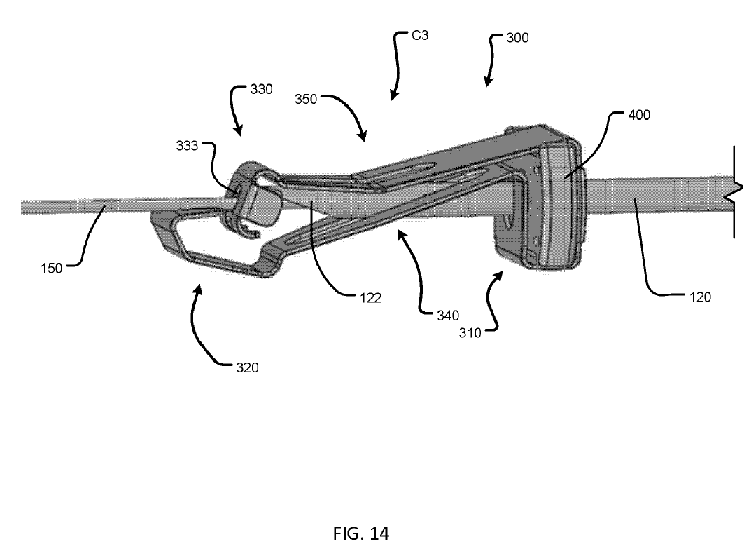

In some implementations, the guidewire 150 is not retracted when the needle 120 is removed. As seen in FIG. 14, the safety clip 300 may be configured to move to an intermediate configuration C3 in which the first flexible arm 340 is moved to a partially closed configuration and the second flexible arm 350 is moved to the fully closed configuration. For instance, during a catheterization procedure, as the needle 120 is proximally retracted from the vessel and the catheter with the guidewire still advanced, the needle tip 122 will similarly move proximally relative to the safety clip 300. As the needle 120 is proximally retracted, the safety clip 300 activates as seen in FIG. 11 and the first wall will begin to slide down the beveled tip 122 of the needle as the needle is further retracted because both the first and second flexible clip arms 340, 350 are biased to move toward each other. Rather than moving fully to the closed configuration C2, as seen in FIG. 12, the safety clip 300 moves to the intermediate configuration C3 in FIG. 14 where the first distal slot 323 and the first distal face 322 abuts the guidewire 150. As the needle 120 is further retracted, the first distal face 322 slides along the surface of the guidewire 150. The guidewire 150 is made of a stiffer material than the safety clip 300 to prevent the guidewire from bending when the first distal face 322 abuts and slides along the guidewire 150 in this intermediate configuration C3. Moreover, when the needle 120 is retracted further, the beveled tip 122 of the needle clears the second distal portion 330 and moves to the fully closed configuration C2.

This is because the second distal slot 333 is configured to receive the guidewire 150 thus allowing the second flexible clip arm 350 to fully close, while the first distal slot 323 is configured to abut the surface of the guidewire 150 allowing the first flexible clip arm 340 to only partially close. In other words, the distal face 322 and/or a portion of the first distal slot 323 continues to abut against the guidewire which prevents the first flexible arm from being biased to its fully closed position. In this intermediate configuration C3, even though the guidewire 150 remains advanced past the first and second distal portions 320, 330 of the safety clip 300, the needle tip 122 remains proximal of both the first and second distal portions, and thus the safety clip 300 is able to provide sharps protection from the needle tip 122.

Further, in this intermediate configuration C3 of the safety clip 300, the guidewire 150 may still be retracted until the first distal face 322 no longer abuts the guidewire, at which point the first flexible arm 340 is biased toward the fully closed configuration C2. Accordingly, the safety clip 300 is configured to provide sharps protection to the needle tip when the guidewire is advanced past the first and second distal portions of the safety clip, and when the guidewire is retracted from the first and second distal portions of the safety clip.

However, even if the guidewire has been retracted prematurely, as the user retracts the needle 120, the first distal wall 321 and the second distal wall 331 of the safety clip 300 will slide down the beveled tip 122 of the needle and activate the first clip arm 340 and the second clip arm to move from the open configuration C1 to the closed configuration C2 as seen in FIG. 12. Thus, after the needle 120 and guidewire 150 is fully removed from the catheter, the first clip arm 340 and the second clip arm 350 will be in the second configuration C2. Additionally, when the first clip arm 340 and the second clip arm 350 are in the second configuration C2, the first proximal aperture 313 may cross over a swaged portion of the needle and the second proximal aperture 314 may then engage the swaged portion of the needle 120 such that the needle 120 is prevented from moving further. Thus, in this second configuration C2, the first distal wall 321 and the second distal wall 331 may be configured to cover a needle tip 122 to protect the user from injury. To form such a cover, the first distal wall 321 is more distal than the second distal wall 331. However, in other aspects of the invention, the second distal wall 331 may be more distal than the first distal wall 321. The first clip arm 340 and the second clip arm 350 do not intersect in either the first configuration C1 or the second configuration C2.

Moreover, when the device is in the closed configuration C2, the first curved elbow 325 and the second curved elbow 335 will also clear the protrusion 103 of the catheter hub 102. At this point, the catheter insertion device 100 and guidewire 150 assembly are ready for removal. The user may also continue to pull back on the catheter insertion device 100. Because the safety clip 300 has activated, the needle 120 and the safety clip 300 are free to be removed from the catheter 101 with the safety clip fully in the closed configuration C2 and covering the tip of the needle.

In embodiments with a safety clip 300 including a keeper 400, pulling back on the catheter insertion device 100 will cause a slight pop to be felt as the keeper 400 is released due to the interference between the keeper 400 and the catheter hub 102. The user may continue to freely pull back the catheter insertion device from the catheter 101 until the needle 120 with its tip covered by the safety clip 300 is entirely removed. At this point, the medical device may be discarded into a sharps bin for disposal, while the catheter 101 is left in the vessel of the patient for treatment.

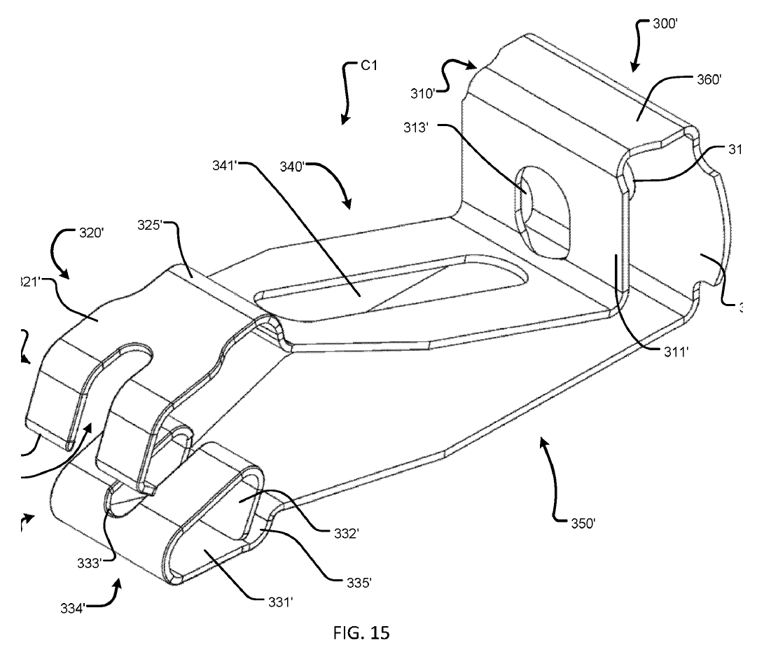

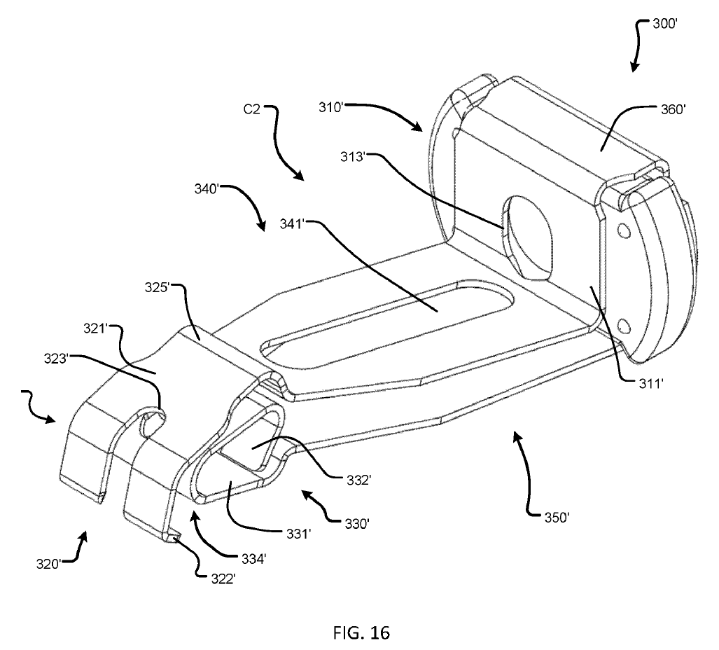

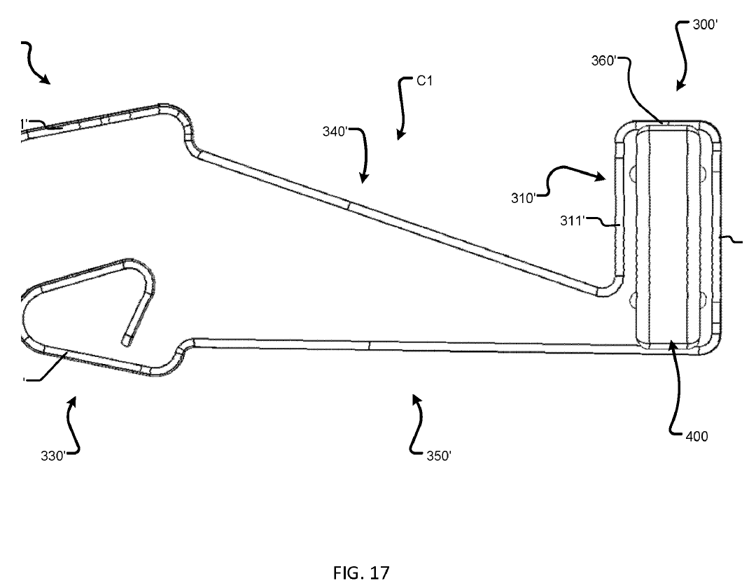

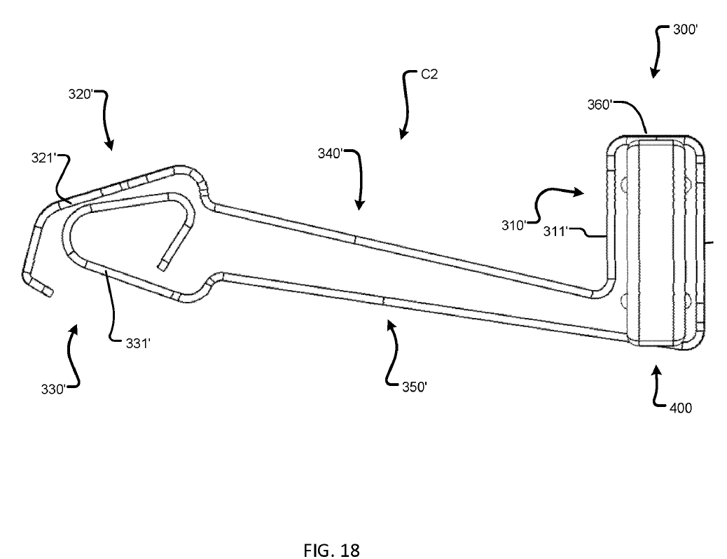

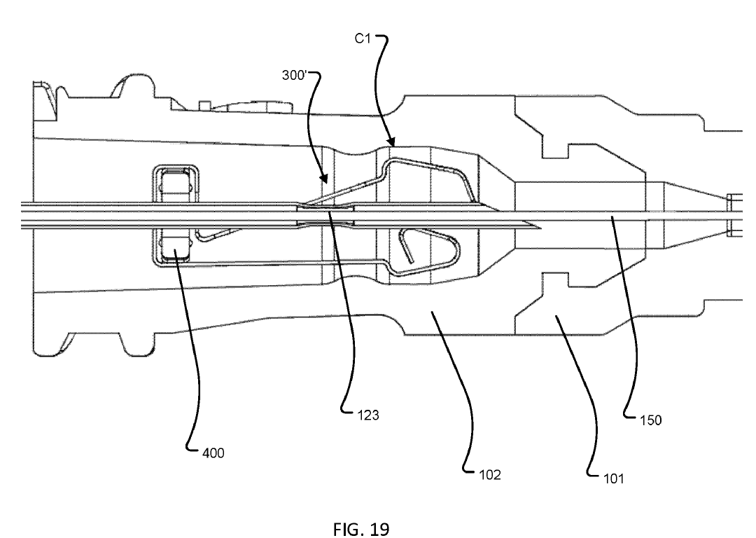

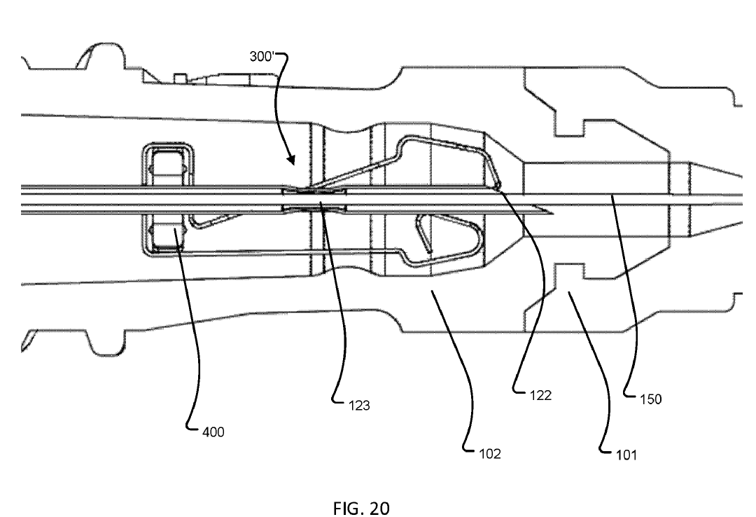

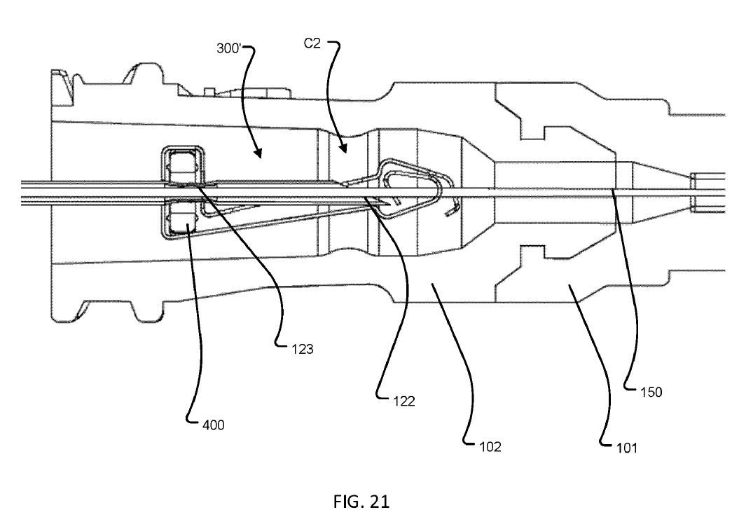

Additionally, when the first clip arm 340 and the second clip arm 350 are in the first configuration C1, the safety clip 300 may exert a first force on the needle 120 tending to return the safety clip 300 back to the second configuration C2. This feature of the safety clip 300 ensures that the safety clip 300 has a resting position intended to cover the needle tip to prevent injury and intended to be disengaged from the catheter hub 102. In contrast, the first configuration C1 ensures the safety clip 300 will actively hold onto the catheter hub 102 while tending to want to move towards the second configuration C2 once the safety clip 300 activates over the needle tip 122. FIGS. 15-21 illustrate a safety clip 300’ according to another aspect of the disclosure. Safety clip 300’ includes a first clip arm 340’ with a single first medial slot 341’ rather than a medial slot on both the first clip arm and the second clip arm, such as seen in FIGS. 1-14. Further, the second clip arm 350’ includes a single medial plane surface rather than a first and second medial plane surface with a medial bend, such as seen in FIGS. 1-13. Moreover, rather than having lateral side walls, the safety clip 300’ includes an elongated second distal curved surface 334’ to provide sharps protection from the entirety of the front of the second clip arm 350’ in the second configuration C2. Safety clip 300’ according to this aspect performs substantially the same as safety clip 300 in FIGS. 1-13 as described above and provides sharps protection when the safety clip 300’ reaches the tip of the needle in the second configuration C2.

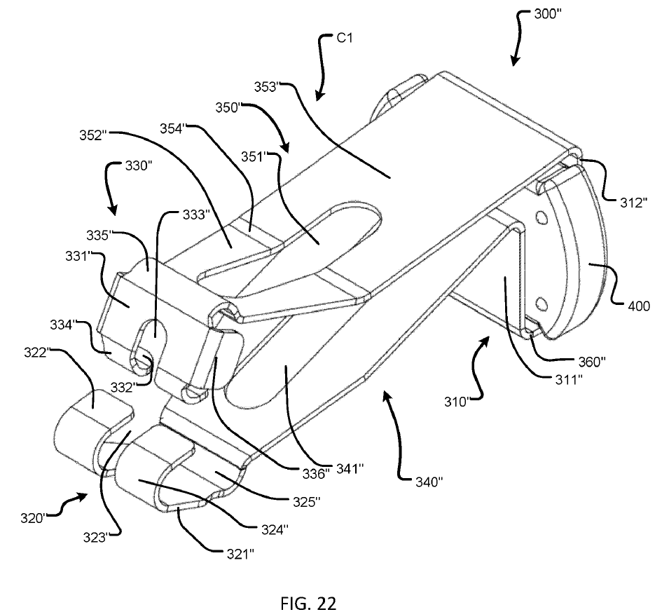

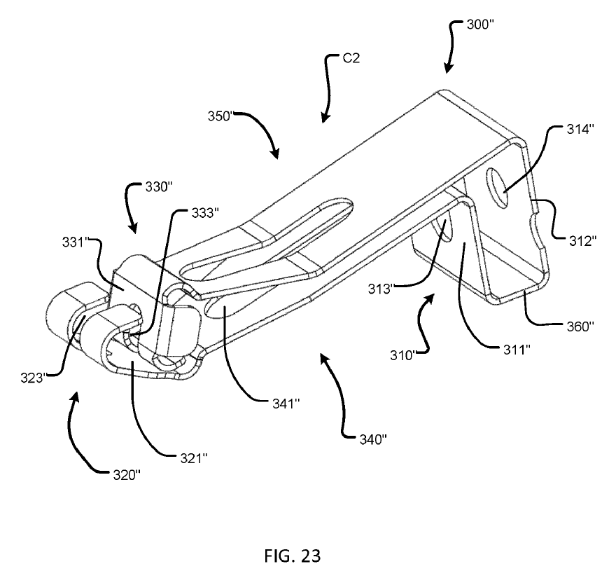

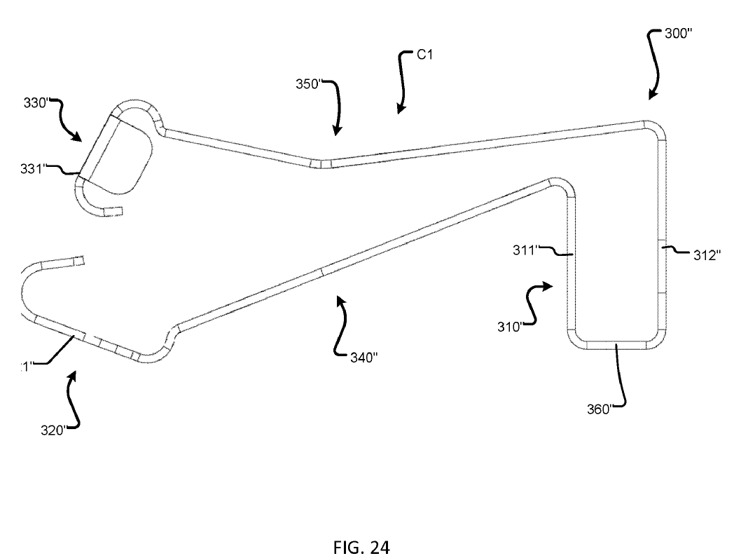

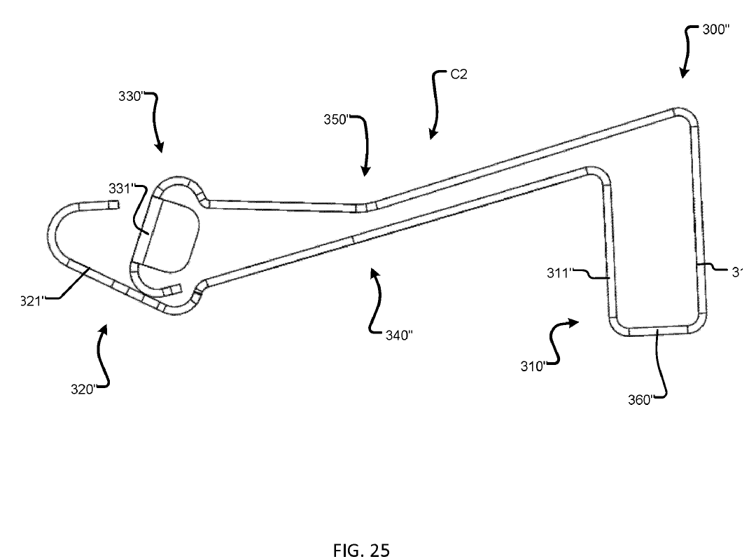

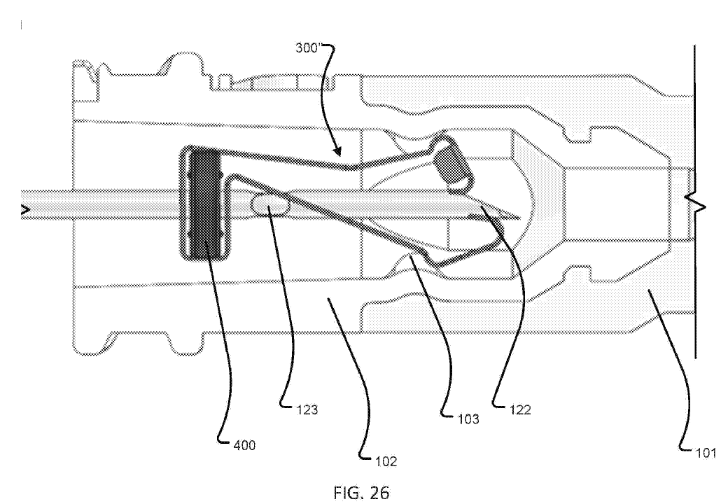

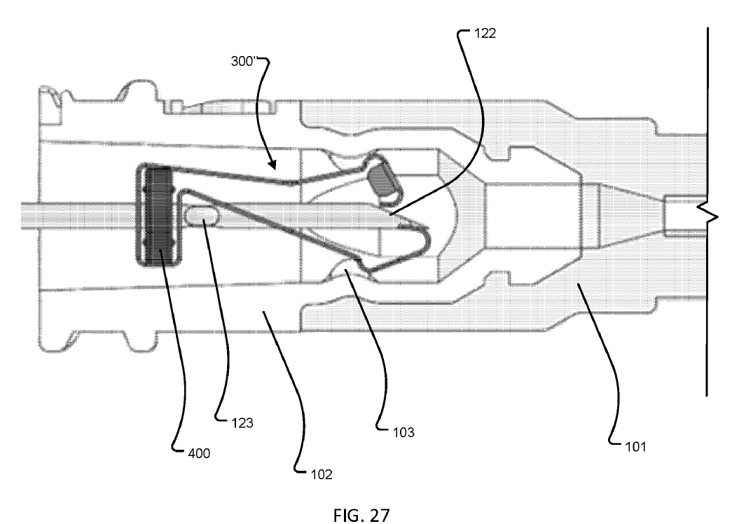

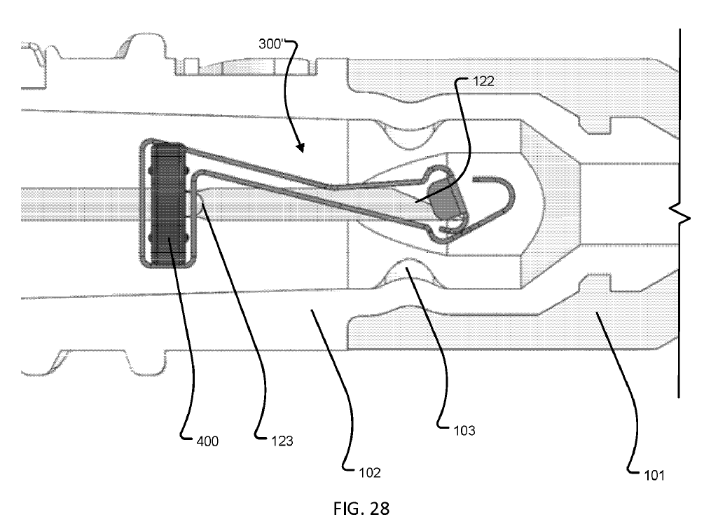

FIGS. 22-28 illustrate a safety clip 300’’ according to another aspect of the disclosure. Safety clip 300’’ includes a second clip arm 350’’ that may move along the top of the needle 120’’ and slide down the beveled tip 122’’ when moving from the first configuration C1 to the second configuration C2 providing more coverage over the beveled tip 122’’ of the needle. Moreover, safety clip 300’’ also has first and second distal slots (323’’, 333’’), which are larger than the guidewire in order to allow the arms of the safety clip 300’’ to activate and reach the closed configuration C2 even when the guidewire is advanced through the needle tip 122’’. As shown in FIGS. 22-28, the safety clip 300’’ according to this aspect otherwise performs substantially the same as safety clip 300 in FIGS. 1-14 as described above and provides sharps protection when the safety clip 300’’ reaches the tip of the needle in the second configuration C2.

The foregoing disclosure provides illustration and description but is not intended to be exhaustive or to limit the implementations to the precise form disclosed. Modifications may be made in light of the above disclosure or may be acquired from practice of the implementations. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A safety clip for a catheter insertion needle, the safety clip comprising:
a proximal portion comprising a first proximal wall directly connected to a second proximal wall at a clip bend, the first proximal wall defining a first proximal aperture and the second proximal wall defining a second proximal aperture, the second proximal wall being more proximal than the first proximal wall;
a first distal portion comprising a first distal wall, the first distal wall defining a first distal aperture for receiving the needle;
a first clip arm extending from the first distal portion to the first proximal wall and comprising a medial aperture for receiving the needle; a second distal portion comprising a second distal wall, the second distal wall defining a second distal aperture for receiving the needle;
a second clip arm extending from the second distal portion to the second proximal wall; the first clip arm and the second clip arm being in contact with the needle, and the first clip arm and the second clip arm are configured to flex between a first configuration and a second configuration to move the safety clip relative to the needle;
wherein, when the first clip arm and the second clip arm are in the first configuration, the safety clip is able to slide along a length of the needle and the first distal wall and the second distal wall are spaced apart;
wherein, when the first clip arm and the second clip arm are in the second configuration, the second proximal aperture is configured to engage a swaged portion of the needle such that the first distal wall and the second distal wall overlap to cover a tip of the needle; and
wherein the first clip arm and the second clip arm do not intersect in either the first configuration or the second configuration.

2. The safety clip according to claim 1, wherein the first and/or second clip arms are at a rest state in the closed configuration.

3. The safety clip according to claim 1, wherein the first clip arm and the second clip arm do not cross when in the open configuration.

4. The safety clip according to claim 1, wherein the first clip arm and the second clip arm do not cross when in the closed configuration.

5. The safety clip according to claim 1, wherein a first distal slot may extend through a portion of the first distal wall and the first distal face, the first distal slot configured to receive a guidewire of the needle, and a second distal slot extending through a portion of the second distal wall and the second distal face, which may be configured to receive the guidewire of the needle; wherein the first distal slot and the second distal slot are configured to allow the first clip arm and the second clip arm to move into the closed configuration with or without the guidewire being advanced beyond the tip of the needle.

6. The safety clip according to claim 1, wherein the safety clip comprises a one-piece body.

7. The safety clip according to claim 1, further comprising a first curved elbow connecting the first clip arm to the first distal wall, and a second curved elbow connecting the second clip arm to the second distal wall.

8. The safety clip according to claim 7, wherein the first and second curved elbows are configured to contact a bump within a hub of a medical device to prevent removal of the safety clip from inside the hub when in the open configuration.

9. The safety clip according to claim 8, wherein the safety clip is configured to be removed from the hub of the medical device when the first clip arm and the second clip arm are in the closed configuration.

10. The safety clip according to claim 1, wherein the safety clip exerts a first force on the needle when the first clip arm and the second clip arm are in the open configuration, which tends to return the safety clip back to the closed configuration.

11. The safety clip according to claim 1, further comprising a keeper disposed between the first and second proximal walls, the keeper configured to provide frictional resistance against an inner surface of a hub of a medical device retain the safety clip inside the catheter hub.

12. The safety clip according to claim 11, wherein the keeper comprises a through-hole configured to slidable receive the needle, wherein a diameter of the through-hole is greater than a diameter of the first proximal aperture and a diameter of the second proximal aperture.

13. The safety clip according to claim 1, wherein the first distal face and the second distal face are configured to press against the needle when the first and second clip arms are in the open configuration.

14. The safety clip according to claim 1, wherein the second distal bend is configured to abut the first distal wall when the first and second clip arms in the closed configuration.

15. The safety clip according to claim 1, wherein a width of the first distal slot is greater than a width of the first medial slot, and a width of the second distal slot is greater than a width of the second medial slot.

16. A method of preventing injury while using a medical device, the method comprising:
providing a safety clip according to claim 1;
positioning the proximal cavity of the hub around the needle and the safety clip and moving the hub proximally until the first distal wall and the second distal wall of the safety clip extend past the bump and within the distal cavity of the hub;
sliding the hub and the safety clip in a proximal direction along a length of the needle such that the first clip arm and the second clip arm transition from the second configuration to the first configuration such that the first distal wall and the second distal wall are spaced apart and the first distal wall and the second distal wall engage with the hub in the distal cavity; and
further sliding the hub and the safety clip in the proximal direction along the length of the needle until the medical device is installed.

17. The method according to claim 16, further comprising moving the hub and the safety clip in a distal direction along the length of the needle until the first distal wall and/or the second distal wall are located at a bevel of the tip of the needle to activate the first clip arm and the second clip arm from the first configuration to the second configuration; and
further retracting the needle in the proximal direction so that the bump is able to pass the first distal wall and the second distal wall of the safety clip and off of the tip of the needle.

18. The method according to claim 16, wherein the safety clip further comprises a keeper configured to maintain the first clip arm and the second clip arm in the second configuration and the first proximal aperture engaged with the swaged portion of the needle in the second configuration.

19. The method according to claim 16, wherein the safety clip further comprises a keeper configured to provide frictional resistance against an inner surface of a hub of a medical device.

20. The method according to claim 16, wherein when the safety clip reaches a bevel of a tip of the needle, the safety clip activates and allows the first clip arm and the second clip arm to move from the first configuration to the second configuration
